(19) United States
(12) Patent Application Publication
(10) Pub. No.: US 2005/0267351 A1
(43) Pub. Date: Dec. 1, 2005

(54) System and method for linking medical information and images

(75) Inventors: Louis M. Humphrey, Durham, NC (US); Bernd Fasel, Herzogenrath (DE); Sanjay Jain, Sugarland, TX (US)

Correspondence Address: SHOOK, HARDY & BACON L.L.P., 2555 GRAND BOULEVARD, KANSAS CITY, MO, 64108-2613, US

(73) Assignee: Cerner Innovation, Inc., Overland Park, KS (US)

(21) Appl. No.: 10/857,506

(22) Filed: Jun. 1, 2004

(51) Int. Cl. A61B 5/05

(52) U.S. Cl. 600/408; 382/156

(57) ABSTRACT

The present invention is directed to a method and system for linking exam information stored in an information system with images stored in a picture archiving system through the use of a study validation server. The method includes receiving notification of receipt of at least one image by the picture archiving system at the study validation server and automatically linking the study to which the received image belongs with exam information from the information system. The method additionally includes directing the information system to send an update reflecting the matching to the picture archiving system.

### CROSS-REFERENCE TO RELATED APPLICATIONS

None.

### STATEMENT REGARDING FEDERALLY SPONSORED RESEARCH OR DEVELOPMENT

None.

### TECHNICAL FIELD

Embodiments of the present invention relate to linking of medical records with medical images. More particularly, embodiments of the invention are directed to facilitating linking of medical records stored in a first system with medical images stored in another system.

### BACKGROUND OF THE INVENTION

Currently, in the hospital environment, imaging data is captured at an image capturing location during a patient examination. The images will later be reviewed by a radiologist at a review station, likely having a location that is not yet determined. Typically, during image capture, patient information related to the images is entered and recorded in an information system such as a radiology information system (RIS). While the patient information is stored in an information system, the captured images are stored in a picture archiving and communication system (PACS), which is a digital library, until a reviewer requests them. Both the RIS and PACS are available through the Cerner™ Corporation and the two systems may be easily integrated by Cerner Corporation. However, other vendors also distribute PACS equipment. When an organization uses a RIS from one vendor and a PACS from another vendor, integration of the two systems may prove problematic. Typical medical information systems and PACS are both expensive systems that hospitals are unlikely to replace. Medical facilities such as hospitals may have invested in a PACS with any number of providers. Accordingly, a solution is needed for integrating the information system and the image archives in a more efficient and accurate manner that does not depend on the particular PACS used by the client.

Currently, in order to coordinate the images and text information in two disparate systems, a technologist retains control over the images and information to complete validation during the acquisition and exam completion process. The technologist is notified of any missing images before completion of the examination and has the opportunity to correct errors using the patient information from the information system. A process may be available to hold images until verified by the technologist, but the technologist is not informed of images having a missing validation. Accordingly, in current systems, images and information are manually related using a health level seven (HL7) standard. Searching two different systems separately creates errors and fails to provide updates.

In some departments such as the radiology department, twenty percent or more of the images may be lost and one third or more of the revenue for work performed may not be billable due to mistakes in identification. The mistakes in identification occur when a stored image is matched with incorrect stored information or is not matched with any stored information. Incorrect identification causes errors in patient care and occasionally results in lack of patient care.

For instance, with current imaging processes, due to inability to match images and information, patients are brought back to the radiology department for re-takes of a particular exam. In some cases, like Computed Tomography (CT), double exposure for the same examination is not healthy. In other cases, a physician may make a diagnosis without a complete and accurate patient health record.

Accordingly, a solution is needed for coordinating information with images to help reduce the number of lost digital images, reduce errors in matching images with information, and reduce patient re-takes due to lack of identification. A solution is also needed that improves the efficiency and accuracy of both patient care and patient billing. Without a completely reliable system for ensuring accurate reflection of information system demographics data on the PACS, the images may still be inaccessible for the patients' examinations in a digital environment. Furthermore, a solution is needed that eliminates manual entry of information and copying of key information from one system to another.

### BRIEF SUMMARY OF THE INVENTION

In one aspect, the present invention is directed to a method for linking exam information stored in an information system with a study containing images stored in a picture archiving and communication system through the use of a study validation server. The method includes receiving notification of receipt of the study by the picture archiving and communication system at the study validation server and automatically linking the study with exam information from the information system. The method additionally includes directing the information system to send an update reflecting the matching to the picture archiving system.

In an additional aspect of the invention, a method is provided for correcting a linkage between exam information and a study containing at least one image stored in a picture archiving and communication system through the use of a study validation server. The method includes receiving a user selection of an unmatch option in order to unmatch the study and the exam information and providing a matching screen display to allow the user to find a correct match. The method additionally includes receiving a user selection of a correct match between the exam information and a selected study and linking the selected study with exam information from the information system.

In an additional aspect of the invention, a study validation system is provided for linking exam information stored in an information system with a study containing images stored in a picture archiving and communication system. The study validation system includes a picture archiving and communication system communication module for receiving notification of receipt of at least one image belonging to the study by the picture archiving and communication system and a linking module for automatically linking the study with exam information from the information system. The study validation system additionally includes an information system communication module for directing the information system to send an update reflecting the matching to the picture archiving and communication system.

In yet an additional aspect of the invention, a system is provided for linking exam information stored in an information system with a study containing images stored in a picture archiving and communication system. The system includes a study validation server having a picture archiving communication module for receiving notification of receipt of at least one image belonging to the study by the picture archiving system, an information system communication module that interacts with the information system, and a linking module capable of automatically linking the study with exam information from the information system. The system additionally includes an archive associated with the study validation server for storing updates to linkages between the study and the exam information.

### BRIEF DESCRIPTION OF THE DRAWINGS

The present invention is described in detail below with reference to the attached drawings figures, wherein:

FIG. 1 is a block diagram illustrating components of a system for linking exam information and image information in accordance with an embodiment of the invention;

FIG. 2 is a block diagram illustrating components of a study validation server in accordance with an embodiment of the invention;

FIG. 3 is a flow chart illustrating work flow for matching information and image data in accordance with an embodiment of the invention;

FIG. 4 is a flow chart illustrating a correction process in accordance with an embodiment of the invention;

FIG. 5 is a screen display illustrating an exam management user interface; and

FIG. 6 is a screen display illustrating a sample match list.

### DETAILED DESCRIPTION OF THE INVENTION

Embodiments of the present invention are directed to a system and method for integrating information from an information system such as RIS with images stored in an image archiving system such as a PACS. In particular, the system disclosed herein is capable of integrating information from a RIS with images from an out-of-system PACS. As will be further explained in conjunction with the FIGs. below, embodiments of the system of the invention include study validation components for performing automatic integration, thereby eliminating manual entry and copying of key information from one system to another.

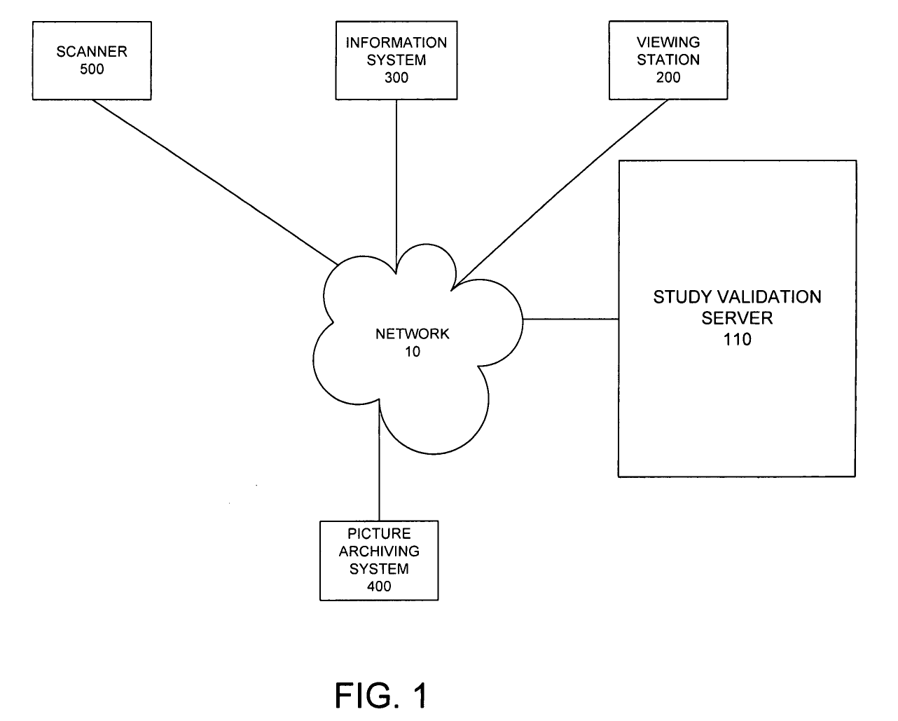

FIG. 1 is a block diagram illustrating a system for linking information with images in accordance with an embodiment of the invention. The system includes multiple components capable of communicating over a network 10. The components may include a study validation server 110, a viewing station 200, an information system such as a RIS 300, a PACS 400, and a scanner 500.

The information system 300 may typically include a RIS. The RIS 300 automates and streamlines information throughout the radiology department, enabling effective process transformation, incorporation of patient registration, order entry, exam tracking, film tracking, transcription, electronic signatures and report distribution.

The scanner 500 may be used interactively with the RIS 300. When evaluating a patient record, a radiologist reviews information from both the RIS 300 and image information captured by a scanner 500. Usually a technologist enters information from the RIS 300 on a console for operating the scanner 500. This may be achieved by either manual entry or by using a DICOM (Digital Imaging and Communications in Medicine) modality work list that provides selectable options. For films, the RIS 300 may generate labels for jackets that contain the films, thereby enabling identification of the films related with exams. CT scanners and other digital scanners no longer require films. Accordingly, the system provides a technique for connecting scanned images in the independent PACS 400 with information from the information system 300.

As set forth above, the PACS 400 stores captured images. The PACS 400 may additionally provide display and web distribution services. The PACS 400 may offer manipulation tools and image analysis tools that can be customized such that protocols automatically display images according to user preferences.

The viewing station 200 may be operable to view images distributed over the web using a system such as Cerner ProVision™ Web. This system offers seamless integration with an electronic medical record and enables image and report availability at the point of care to provide a caregiver with up-to-date information.

In embodiments of the invention, the study validation server 110 may include an archive for storage, which will be Cerner ProVision™ PACS. In other embodiments, DICOM is used instead of the archive. Furthermore, in embodiments of the invention the study validation server 110 may be incorporated in the RIS 300 and may direct the flow of communication between the RIS 300 and the archive.

The study validation server 110 accesses demographic and exam information in the information system 300, automatically validates image information received from DICOM modalities, and corrects inaccurate information. The study validation server 110 serves as the sentinel between the information system 300 and the PACS 400 by safeguarding critical data, creating an accurate link between PACS images and RIS information, and enabling a uniquely effective person-centric approach to image management. The study validation server 110 allows the information system 300 to monitor and validate data sent to the PACS 400, as will be further explained below in relation to the method of the invention, thereby ensuring data integrity.

The study validation server 110 can be used in conjunction with any PACS 400 that support the standard communication protocol DICOM. The study validation server 110 is capable of providing a central source of digital study information. Furthermore, the study validation server 110 can increase productivity by leveraging user-defined search criteria to automatically match RIS exam information with DICOM studies. The user defines this criteria in RIS 300 which can be generic for the entire department or specific to type of scanner (CT, MR, etc.) or specific to each particular scanner. The use of the study validation server 110 reduces errors by synchronizing patient and study information between the RIS 300 and the PACS 400. The study validation server 110 automates verification of acquired image information before transferring images to the PACS 400 for optimal workflow.

For digital images, the study validation server 110 performs several functions during the exam completion process used by the technologist. First, the study validation server 110 updates demographic headers of the digital images with correct information from the RIS 300. Secondly, the study validation server 110 corrects any errors made during typing of the information on the modality console. Thirdly, the study validation server 110 resolves erroneous selection of patient and procedures from the modality work list. Finally, the study validation server 110 enables the electronic medical record to access images in addition to textual information from the RIS 300.

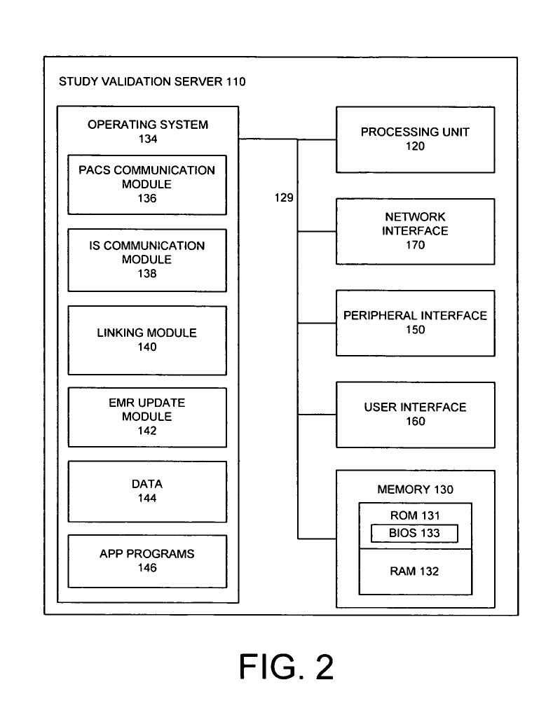

FIG. 2 illustrates the components of the study validation server 110 in accordance with an embodiment of the invention. The study validation server 110 may include a processing unit 120, a peripheral interface 150, a user interface 160, and a network interface 170. The study validation server 110 may also include a memory 130. A system bus 129 may be used to couple the aforementioned components.

The system memory 130 may include computer storage media in the form of volatile and/or nonvolatile memory such as read only memory (ROM) 132 and random access memory (RAM) 140. A basic input/output system 133 (BIOS), containing the basic routines that help to transfer information between elements within the study validation server 110, such as during start-up, is typically stored in ROM 132. RAM 134 typically contains data and/or program modules that are immediately accessible to and/or presently being operated on by processing unit 120.

The study validation server 110 may include a PACS communication module 136 for communicating with the PACS 400 and an information system (IS) communication module 138 for communicating with the RIS 300. It further includes a linking module 140 for linking information to be stored in the PACS 400 with information from the RIS 300. The study validation server 110 may further include an electronic medical record (EMR) update module 142 for updating electronic medical records pertinent to each patient. It may include, application programs 146, and data 144.

The PACS communication module 136, the IS communication module 138, the linking module 140, the EMR update module 142, and application programs 146 may be described in the general context of computer-executable instructions, such as program modules, being executed by a computer. Generally, program modules include routines, programs, objects, components, data structures, etc. that perform particular tasks or implement particular abstract data types. Moreover, those skilled in the art will appreciate that the invention may be practiced with other computer system configurations, including multiprocessor systems, microprocessor-based or programmable consumer electronics, minicomputers, mainframe computers, and the like.

The study validation server 110 may also include other removable/non-removable, volatile/nonvolatile computer storage media. A hard disk drive may be provided that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive is typically connected to the system bus through a non-removable memory interface and magnetic disk drive and optical disk drive are typically connected to the system bus by a removable memory interface.

A user may enter commands and information through the user interface 126 using input devices such as a keyboard and pointing device, commonly referred to as a mouse, trackball or touch pad. Other input devices may include a microphone, satellite dish, scanner, or the like. These and other input devices are often connected to the processing unit 120 through a user input interface 160 that is coupled to the system bus 129, but may be connected by other interface and bus structures, such as a parallel port or a universal serial bus (USB). A monitor or other type of display device and other peripherals may also be connected to the system bus 129 via an interface, such as the peripheral interface 150.

The illustrated study validation server 110 is merely an example of a suitable environment for the system of the invention and is not intended to suggest any limitation as to the scope of use or functionality of the invention. Neither should the study validation server 110 be interpreted as having any dependency or requirement relating to any one or combination of components illustrated.

The study validation server 110 in the present invention may operate in a networked environment using logical connections to communicate with networked components. Logical connections for networking may include a local area network (LAN) or a wide area network (WAN), but may also include other networks. When used in a LAN networking environment, the system may be connected to the LAN through the network interface 170 or adapter. When used in a WAN networking environment, the study validation server 110 typically includes a modem or other means for establishing communications, such as the Internet. The modem, which may be internal or external, may be connected to the system bus 129 via the user input interface 160 or other appropriate mechanism.

Although only the study validation server 110 has been described in detail with respect to the appropriate computerized environment, it should be understood that the other components shown in FIG. 1, such as the RIS 300 and the PACS 400 operate in a similar computerized environment.

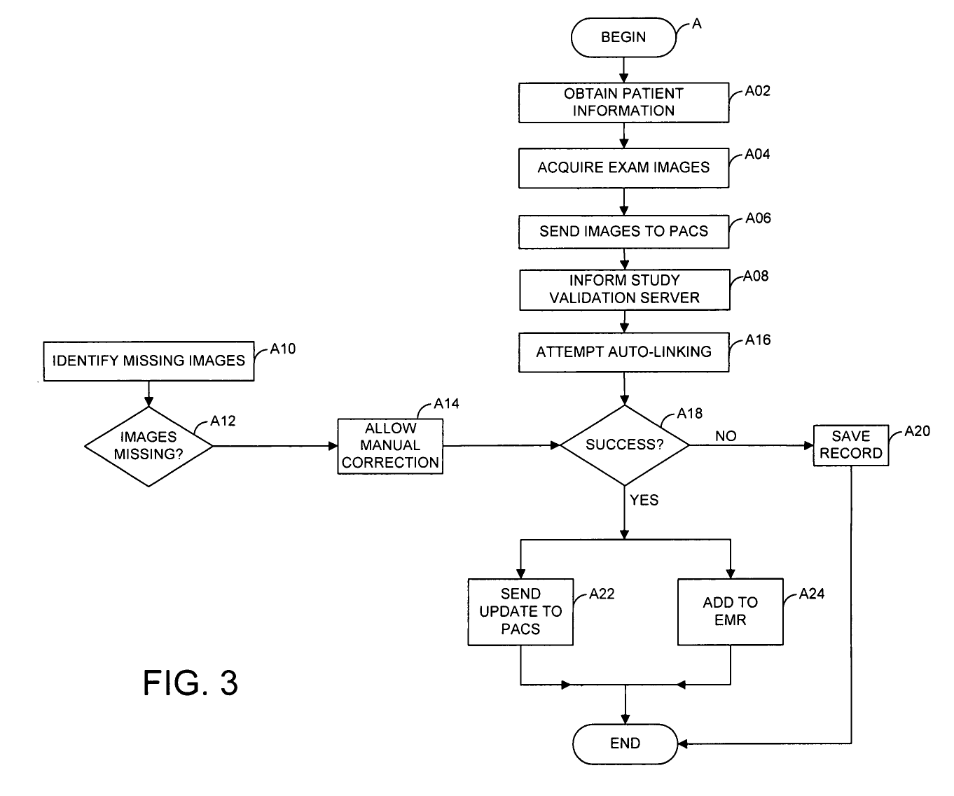

FIG. 3 is a flow chart illustrating a study validation process in accordance with an embodiment of the invention. In step A02, after a patient has been routed to an exam room, a technologist acquires patient information. If a scanner accessible to the technologist is capable of obtaining information directly from the RIS 300 to be used for image acquisition, the technologist selects an exam from the list on the scanner. In the absence of that capability, the technologist will enter the information manually in the fields provided by the scanner display. In step A04, the technologist acquires exam images. In step A06, the scanner 500 sends the acquired images to the PACS 400. In step A08, the PACS 400 announces the arrival of the images to the study validation server 110.

As an alternative to step A08, the receiving PACS 400 may forward the images to an archive, which may be a Cerner ProVision PACS, for study validation. Either technique results in the announcement of the storage of the images to the study validation server 110.

In step A16, the study validation server 110 through the linking module 140, attempts to automatically link the study from the PACS 400 with the information in the RIS 300. If automatic linking succeeds in step A18, the RIS 300 sends the information in step A20 to the PACS 400 in the form of an health level seven (HL7) update to correct or modify the demographics and exam information for the images while updating the contents of the Cerner ProVision™ PACS or other archive associated with the study validation server 110.

If the automatic linking does not succeed in step A18, the RIS 300 saves the record in step A20. The saved record will be eligible for manual matching as described below.

When technologist completes an exam, the study validation server 110, determines if the exam has study matched to it or not. If no study is matched, the study validation server 110 warns the technologist to match before proceeding. If study does not exist in step A12, the study validation server allows the technologist to manually match them in step A14.

Furthermore, simultaneously with the update in step A22, the study identification facilitated by the study validation server 110 is added to the orders in the RIS 300 and results in an electronic medical record (EMR). The EMR is updated with an image link from the PACS 400. Thus, the caregivers, including physicians, nurses, etc. have access to patient textual information and digital images of the exams at the same time for optimal care. This step enables viewing of the images stored in the temporary archive at the viewing station 200 through an image distribution system such as Cerner ProVision™ Web.

Accordingly, digital studies acquired during examination in an environment such as the radiology department are linked with exams in the information system 300 while being saved on the PACS 400 with correct demographics and are retrievable for reading and viewing by the interested and authorized users.

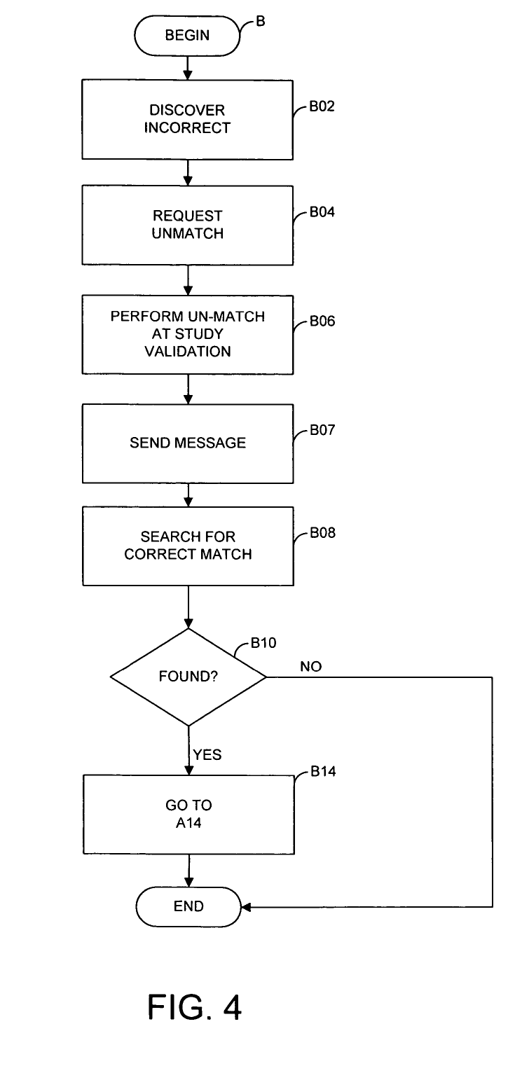

FIG. 4 is a flowchart illustrating a process for invalidating match or unmatching information and images. In step B02, an incorrect match is discovered by the technologist at the completion of the patient's examination in the RIS 300 or later. In step B04, the technologist requests an unmatching. In step B06, the study validation server 110 performs the unmatching using its linking module 140. In step B07 a message is sent upon completion of unmatch. In step B08, the technologist searches for the correct match. If the correct match is found in step B10, the process continues by proceeding to step A14, where linking is attempted manually.

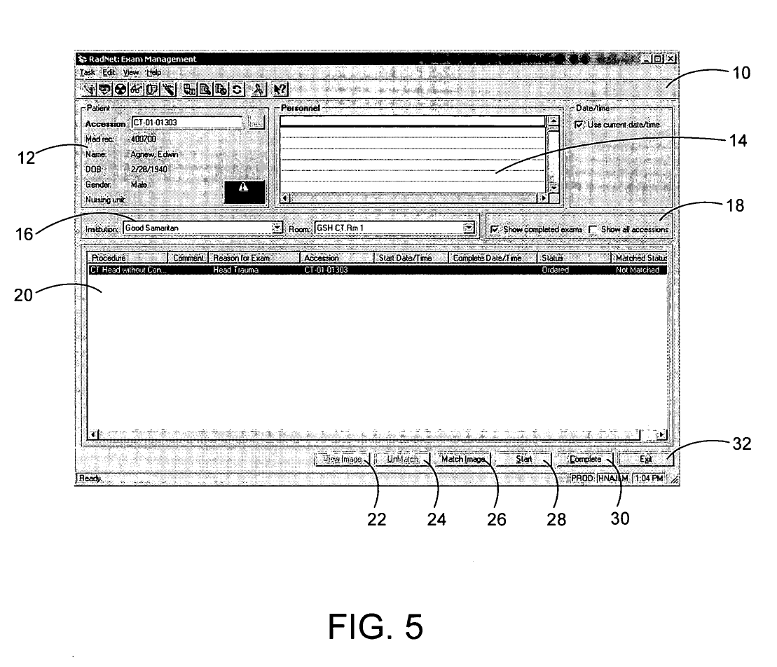

FIG. 5 illustrates an exam management screen display 10 available to the technologist. The screen display 10 may be accessible to the technologist through the user interface of the RadNet Millennium applications and is provided through the study validation server 110. The screen display 10 may include patient information 12. The patient information 12 may include such details as an accession number, a medical record number, a name, a date of birth, and a gender. A personnel space 14 may be adjacent the patient information 12. An institution information section 16 may include the name of the institution and a room number. Checkboxes 18 may allow selectable items to be shown on the screen display such as completed exams and accessions. An exam information section 20 provides exam information matched with the accession code, a status, and an indication of whether the exam has been matched. Selectable options include a view image option 22, an unmatch option 24, a match image option 26, a start option 28, a complete option 30, and an exit option 32. By selecting an option such as the unmatch option 24, the technologist is able to complete steps B04 and B06 shown above with respect to FIG. 4. By selecting the match option 26, the technologist is able to search for the correct match is shown in step B08 of FIG. 4.

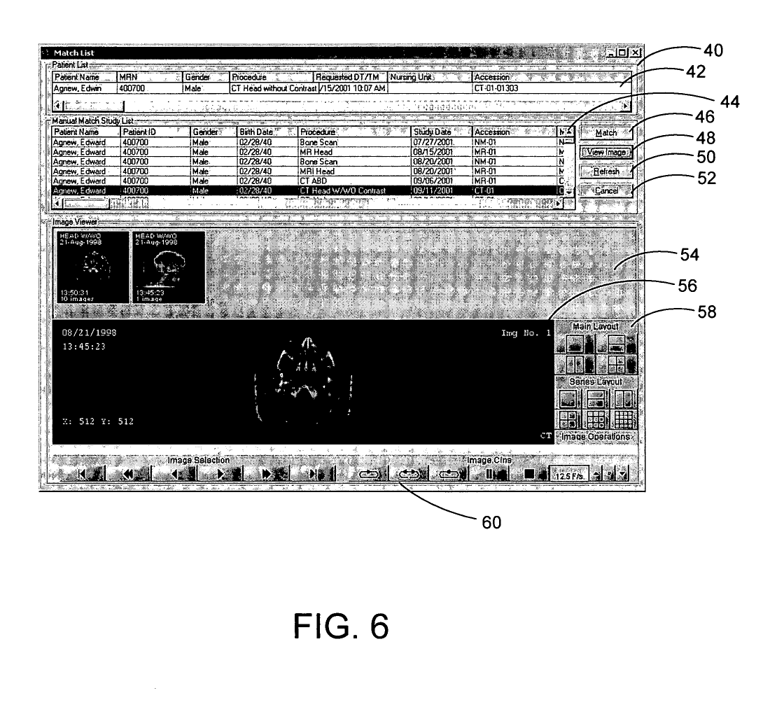

FIG. 6 illustrates a match list screen display 40 that is provided in conjunction with step B08 in accordance with an embodiment of the invention. A patient list 42 includes patient information. The patient list 42 is followed by a manual match study list 44. Selectable options include a match option 46, a view image option 48, a refresh option 50, and a cancel option 52. An image views section 54 shows multiple available image views. An enlarged selected image 56 may be provided. The user may select the layout for viewing images with selectable options 58. Further selectable options 60 are also provided.

The study validation components provide a methodology to reliably link digital images with radiology exams and allow correction of information on the images stored in the PACS. The study validation server is a solution for integrating the RIS and PACS. It extends integration between RIS and PACS beyond what present standards can provide. This process is developed to be a part of the normal workflow of the technologist and accomplished through the technologist's interaction with the RIS. The system eliminates manual entry and copying of key information from one system to another. The invention enables use of an external or out-of-system PACS to provide digital storage of images, while the workflow functions are governed by the RIS.

While particular embodiments of the invention have been illustrated and described in detail herein, it should be understood that various changes and modifications might be made to the invention without departing from the scope and intent of the invention.

From the foregoing it will be seen that this invention is one well adapted to attain all the ends and objects set forth above, together with other advantages, which are obvious and inherent to the system and method. It will be understood that certain features and sub-combinations are of utility and may be employed without reference to other features and sub-combinations. This is contemplated and within the scope of the appended claims.

1. A method for linking exam information stored in an information system with a study containing at least one image stored in a picture archiving system through the use of a study validation server, the method comprising:
receiving notification of receipt of at least one image by the picture archiving system at the study validation server;
automatically linking the study containing the at least one received image with exam information from the information system; and
directing the information system to send an update reflecting the matching to the picture archiving system.

2. The method of claim 1, further comprising directing the information system to send the update reflecting the matching to an archive associated with the study validation server.

3. The method of claim 1, further comprising allowing a user to identify a missing study for the exam information.

4. The method of claim 3, further comprising providing a screen display for allowing manual correction.

5. The method of claim 1, further comprising incorporating the update in an electronic medical record.

6. The method of claim 1, further comprising receiving a user selection of an unmatch option in order to delete an association between the study and exam information.

7. The method of claim 6, further comprising providing a matching screen display to allow the user to find a correct match.

8. The method of claim 7, further comprising receiving a user selection of a correct match between the exam information and a selected study.

9. A method for correcting a linkage between exam information and a study stored in a picture archiving system through the use of a study validation server, the method comprising:
receiving a user selection of an unmatch option in order to unmatch the study and the exam information;
providing a matching screen display to allow the user to find a correct match;
receiving a user selection of a correct match between the exam information and a selected study; and
automatically linking the selected study with exam information from the information system.

10. The method of claim 9, further comprising directing the information system to send an update reflecting the matching to the picture archiving system.

11. The method of claim 9, further comprising directing the information system to send an update reflecting the matching to an image archive associated with the study validation server.

12. The method of claim 9, further comprising storing the update in an electronic medical record.

13. The method of claim 9, further comprising warning the user of a missing matched study upon completion of an exam in the information system.

14. The method of claim 13, further comprising allowing the user to manually review the study and match the study with information upon receiving the warning.

15. A study validation system for linking exam information stored in an information system with a study including images stored in a picture archiving system, the study validation system comprising:
a picture archiving system communication module for receiving notification of receipt of at least one image belonging to the study by the picture archiving system;
a linking module for automatically linking the study with exam information from the information system; and
an information system communication module for directing the information system to send an update reflecting the matching to the picture archiving system.

16. The system of claim 15, wherein the information system communication module directs the information system to send the update reflecting the matching to an archive associated with the study validation server.

17. The system of claim 15, wherein the information system communication module further comprising allows a user to identify a missing study.

18. The system of claim 17, wherein the information system communication module comprises tools for providing a screen display for allowing manual correction.

19. The system of claim 15, further comprising an electronic medical record updating module for incorporating the update in an electronic medical record.

20. The system of claim 15, wherein the linking module receives a user selection of an unmatch option in order to delete an association between the study and exam information.

21. The system of claim 20, wherein the linking module further comprises tools for providing a matching screen display to allow the user to find a correct match.

22. The system of claim 21, wherein the linking module receives a user selection of a correct match between the exam information and the selected study.

23. A system for linking exam information stored in an information system with a study stored in a picture archiving system, the system comprising:
a study validation server comprising a picture archiving communication module for receiving notification of receipt of at least one image by the picture archiving system, an information system communication module that interacts with the information system, and a linking module capable of automatically linking the at least one received image with exam information from the information system; and
an archive associated with the study validation server for storing updates to linkages between the study and the exam information.

24. The system of claim 23, further comprising an electronic medical record update module for updating an electronic medical record.
